(19) United States
(12) Patent Application Publication
(10) Pub. No.: US 2003/0171329 A1
(43) Pub. Date: Sep. 11, 2003

(54) Stable compositions comprising tezacitabine

(76) Inventors: Richard E. Jones, Solana Beach, CA (US); Mike T. Li, Cupertino, CA (US); Andy Trammel, Olathe, KS (US); Christine M. Harrison-Bowman, Overland Park, KS (US); Kimberly R. Shirley, Thornton, CO (US)

Correspondence Address: Chiron Corporation, Intellectual Property, P.O. Box 8097, Emeryville, CA, 94662-8097, US

(21) Appl. No.: 10/365,896

(22) Filed: Feb. 12, 2003

Related U.S. Application Data
(60) Provisional application No. 60/357,185, filed on Feb. 15, 2002.

(51) Int. Cl. A61K 31/7072

(52) U.S. Cl. 514/49

(57) ABSTRACT

The present invention relates to tezacitabine ((E)-2′-fluoromethylene-2′-deoxycytidine) formulations which are stable for long periods of time. The present invention further relates to methods of formulating stable tezacitabine formulations.

This application claims priority to U.S. provisional application Serial No. 60/357,185, filed Feb. 15, 2002.

### BACKGROUND OF THE INVENTION

1. Field of the Invention

This invention relates to stable aqueous compositions comprising tezacitabine ((E)-2′-fluoromethylene-2′-deoxycytidine), a known anti-cancer nucleoside analog. This invention further relates to methods of preparing and storing aqueous compositions so that these compositions exhibit prolonged shelf-life.

References

The following references are cited herein as superscript numbers:

¹ Notari, R. E.; Chin, M. L.; Wittlebort, R., “Arabinosylcytosine Stability in Aqueous Solutions: pH Profile and Shelflife Prediction”, J Pharm Sci 1972 61(8), 1189-1196.

² Anliker, S. L.; McClure, M. S.; Britton, T. C.; Stephan, E. A.; Maple, S. R.; Cooke, G. G., “Degradation chemistry of gemcitabine hydrochloride, a new antitumor agent”, J. Pharm. Sci. 1994, 83(5), p 716-719.

³ Takahashi, T.; Nakashima, A.; Kanazawa, J.; Yamaguchi, K.; Akinaga, S.; Tamsoki, T.; Okabe, M., “Metabolism and ribonucleotide reductase inhibition of (E)-2′-deoxy-2′-(fluoromethylene)cytidine, MDL 101,731, in human cervical carcinoma HeLa S3 cells”, Cancer Chemother. Pharmacol., 1998, 41 pp 268-274.

⁴ Zhou, Y.; Achanta, G.;Pelicano, H.; Gandhi, V.; Plunket,t W.; Huang, P., “Action of (E)-2′-deoxy-2′-(fluoromethylene)cytidine on DNA metabolism: Incorporation, excision, and cellular response. Mol. Pharmacol., 2002, 61(1) pp 222-229.

⁵ Woessner, R. D.; Bitonti, A. J., “FMdC—antineoplastic ribonucleotide-diphosphate reductase inhibitor”, Drugs Fut., 1999, 24(5) pp 502-510.

All of the above references are herein incorporated by reference in their entirety to the same extent as if each individual reference was specifically and individually indicated to be incorporated by reference in its entirety.

2. State of the Art

Tezacitabine ((E)-2′-deoxy-2′-(fluoromethylene)cytidine, or FMdC) is an anticancer nucleoside analog having the following structure:

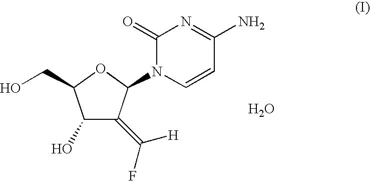

It is structurally similar to deoxycytidine, a naturally occurring nucleoside that is one of the building blocks of DNA. Tezacitabine consists of cytosine (the heterocyclic purine ring) linked to a substituted sugar 5-membered ring.

Nucleoside analogs are a class of drugs that affect DNA synthesis that have traditionally been used to treat hematologic cancers, such as leukemia. More recently, a novel nucleoside analog, gemcitabine, has been found useful in the treatment of solid tumors. Tezacitabine has likewise exhibited activity in animal studies against a broad spectrum of human and rodent tumors, including tumors of the lung, colon, breast (both estrogen-dependent and estrogen-independent), prostate, and pancreas as well as leukemias and lymphomas. Tezacitabine has been shown to be well tolerated in human patients with solid tumors treated in Phase I clinical trials.

When administered to patients, tezacitabine enters into cancerous cells where it is metabolized into two active forms that interrupt DNA synthesis: tezacitabine diphosphate and tezacitabine triphosphate. The diphosphate inhibits ribonucleotide reductase and the triphosphate is incorporated directly into the replicating strand of DNA where it causes premature chain termination.³⁻⁵

Tezacitabine, as with many anticancer drugs, is typically administered by intravenous injection or infusion of a physiologically compatible aqueous solution of the drug. The administration schedule can be on a frequent basis, e.g., daily for 5 days followed by a 1 to 2 week rest, then repeat, or can be on a less frequent basis, e.g., once every three weeks. The dose intensity in humans ranges from about 2 mg/m²/week to about 1000 mg/m²/week. Dosage is heavily schedule-dependent: when administered daily for 5 days, the lower dose range is appropriate (e.g., about 0.5 to 10 mg/m² per injection), whereas when administered less frequently, e.g., once every 2 to 3 weeks, a higher dose range is appropriate (e.g., 200 to 1000 mg/m² per injection).

The drug is typically presented to health care personnel as an aqueous solution or as a powder for reconstitution just before use. The solution or reconstituted solution can be administered directly to the patient by bolus injection or slow infusion, or alternatively can be diluted in standard intravenous solutions (e.g., 0.9% sodium chloride injection, 5% dextrose injection, lactated Ringer's solution, etc.) and infused over a period of time. The injected or infused solution must be physiologically compatible, i.e. one that is sterile, nonpyrogenic, generally isosmotic, and within a physiologically compatible pH range, e.g., between about pH 5 and pH 9. If the solution is administered slowly, a wider range of pH and osmolality is acceptable, due to the rapid dilution as it enters the blood stream.

The commercial viability of the drug product presented to the health care personnel generally requires a minimum shelf life of eighteen months when stored at room temperature (20-25° C.) or under refrigeration (2-8° C.). The shelf life is typically defined as the storage time after manufacture of the drug product during which the drug experiences no more than about 5 to 10% degradation. In a preferred embodiment, such injectable drugs are provided as readily injectable solutions and are stored at ambient temperatures. Contrarily, drugs lacking acceptable shelf-life in aqueous solution at ambient or refrigerated conditions can instead be formulated as lyophilized (freeze-dried) compositions that are reconstituted into solutions at the time of use. Because the drug is present in a dry state, such lyophilized compositions are generally more stable than solutions.

Lyophilized formulations are, however, much less desirable than aqueous compositions, for a number of reasons. First the extra step of reconstitution in the hands of the user is a drawback, particularly when cytotoxic agents like tezacitabine and other anticancer drugs are involved. Not only is the procedure time-consuming, but the practitioner risks additional occupational exposure to the drug. Second, the manufacturing process development for lyophilized formulations is more difficult and involved than the simple manufacture of solutions. And finally the manufacture of these compositions is considerably more expensive relative to aqueous formulations. Solution formulations, then, are easier to use, generally safer for health care personnel, and less difficult and less expensive to manufacture.

Tezacitabine is quite unstable as a simple aqueous solution of drug. Specifically, when the drug is stored at room temperature in an unbuffered aqueous solution, the solution changes from colorless to yellow to light brown within five days, indicative of degradation. Rapid degradation is also observed at temperatures slightly above room temperature: at 35° C. greater than 90% loss of drug was observed (using an HPLC method to determine amount of intact, unreacted drug) within 4 days, along with a large drop in pH (from about 7.2 to about 4.3). A simple aqueous solution of drug in water is clearly inadequate in terms of providing acceptable long-term stability.

### SUMMARY OF THE INVENTION

The present invention provides for an aqueous composition comprising water, tezacitabine, and a buffer. The buffer maintains the pH of the tezacitabine composition at about 7 or above.

Another aspect of this invention provides for methods for enhancing the shelf life of an aqueous solution of tezacitabine. The method for extending the shelf life comprises preparing an aqueous solution of tezacitabine and combining a sufficient amount of a buffer to the tezacitabine solution to maintain the pH of the resulting solution at at least 7 or above.

In preferred embodiments, the buffer maintains the pH between 8 and 11, and more preferably between 8.5 and 9.0. In another embodiment, the buffer is a pharmaceutically acceptable buffer, such as phosphate, glycine, glutamate, or TRIS (2-amino-2-hydroxymethyl-1,3-propanediol, or tromethamine). In yet another embodiment the concentration of tezacitabine in the composition is about 0.5 mg/mL to about 80 mg/mL. In still another embodiment, the water is sterile water. In still another embodiment the method further comprises storing the liquid composition at a temperature below about 40° C., more preferably about 25° C. or below.

### DETAILED DESCRIPTION OF THE INVENTION

This invention provides for aqueous compositions of tezacitabine having long-term shelf life under ambient storage conditions. Specifically, the compositions of this invention comprise tezacitabine and a buffer wherein the buffer employed provides for an aqueous solution have a pH of 7 or greater. Surprisingly, when so buffered, the shelf life of the composition is significantly greater than unbuffered compositions or buffered compositions having a pH of less than about 7. The data in Example 1 demonstrate that the drug is very unstable at low pH, but is increasingly more stable as the pH is raised to 7 or 9.

The stability of tezacitabine at high pH is unexpected, especially when compounds of similar structure are evaluated. Structurally, tezacitabine closely resembles cytarabine, whose degradation has been characterized.¹ Cytarabine is most stable at neutral pH (7-8), and is much less stable at lower pH as well as higher pH. Tezacitabine also closely resembles gemcitabine, whose degradation has also been characterized.² Gemcitabine is relatively stable in acid conditions (low pH), but is

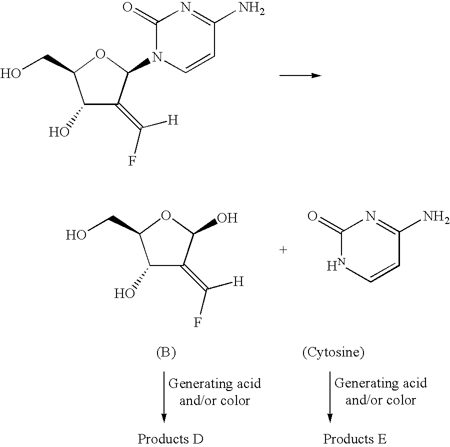

considerably less stable at high pH.

The major degradation product of tezacitabine seen in HPLC chromatograms of degraded samples is cytosine. Based on this, but without limitation to any specific theory, the mechanism of degradation is believed to involve hydrolysis of the glycosidic bond between the purine ring and the sugar ring. This hydrolysis reaction is believed to occur rapidly at low pH but much less rapidly at high pH. The liberated sugar moiety is believed subsequently to undergo rapid degradation to a spectrum of products, one or more of which are highly acidic as well as being colored. The cytosine may also further degrade to acid-producing and/or color-producing species. The degradation, therefore, has a propensity to become autocatalytic, as the liberated acid species would tend to lower the pH, thus accelerating the degradation of previously undegraded tezacitabine molecules.

The upper limit of the concentration of tezacitabine in these formulations is determined by the solubility of the drug in the desired buffer system. Preferably the concentration ranges from 0.5 mg/mL to 80 mg/mL.

Degradation of tezacitabine can readily be monitored by the the use of high pressure liquid chromatography (HPLC) methods which separate the drug from its degradation products and quantitate the amount of intact drug remaining in the solution. Typically such studies are performed by filling aliquots of various tezacitabine solutions into appropriate containers (e.g., stoppered glass vials or sealed ampoules) and storing these samples for various periods of time under various storage conditions. Although the storage conditions of interest are at ambient room temperature (20-25° C.) or refrigeration (2-8° C.), the time required to establish stability over 18 months or more is impractical for early formulation design work. Typically such stability studies are carried out at higher temperatures, where degradation is expected to occur more rapidly. Various formulations can be compared as to relative stability under such conditions. Also, kinetic models such as the Arrhenius equation can be used to predict lower-temperature stability behavior from results at the accelerated conditions. Example 2 below provides such an analysis on a particular embodiment of the invention. Example 3 below confirms the desired 18+-month stability at 25° C. on another embodiment of the invention. And Examples 2 and 4 provide examples of a wide range of physiologically compatible buffer systems appropriate for use in the invention.

### EXAMPLES

In these examples, unless otherwise indicated, all temperatures are in degrees Celcius and all percentages are weight percentages based on the total weight of the composition. Similarly, the following abbreviations are employed herein and are as defined below. Unless defined, the abbreviations employed have their generally accepted meaning:

HPLC=high performance liquid chromatography

M=molar

mM=millimolar

mg=milligram

mL=milliliter

nm=nanometer

μg=microgram

μL=microliter

μm=micron

v/v=volume to volume

v/v/v=volume to volume to volume

### Example 1

In this example the effects of pH and storage temperature on the stability of tezacitabine (FMdC) solutions was evaluated. FMdC solutions were made up at 1 mg/mL in aqueous sodium phosphate buffers at pH 1, 3, 5, 7 and 9, then filled in sealed glass ampoules and placed at 5°, 25°, and 45° C. FMdC content was assessed at various times using an HPLC method. The column was a PACK ODS column (YMC, Inc, Wilmington, Del.) with a mobile phase of 55/40/5 (v/v/v) 0.086M NaH₂PO₄ (pH 7)/0.05M 1-pentanesulfonic acid sodium salt/acetonitrile. Detection was by UV at 268 nm. Flow rate was 1.5 mL/min and injection volume was 10 μL. Results are shown in Table 1 below.

TABLE 1
Degradation of FMdC at Different Storage Temperatures

| | 5° C. | 25° C. | 45° C. |
|---|---|---|---|
| Temperature | FMdC content | FMdC content | FMdC content |
| Time (days) | (% of initial) | (% of initial) | (% of initial) |
| pH 1 | | | |
| 0 | 100.0 | 100.0 | 100.0 |
| 1 | 93.2 | 45.7 | 3.9 |
| 7 | | 4.5 | |
| 14 | 70.0 | | |
| 29 | 47.1 | | |
| 0 | 100.0 | 100.0 | 100.0 |
| 1 | 89.4 | 50.5 | 4.4 |
| 7 | | 4.7 | |
| 14 | 73.3 | | |
| 29 | 51.7 | | |
| pH 5 | | | |
| 0 | 100.0 | 100.0 | 100.0 |
| 7 | 95.8 | 73.0 | 2.5 |
| 14 | 98.2 | 56.3 | |
| 29 | 93.5 | 28.6 | |
| pH 7 | | | |
| 0 | 100.0 | 100.0 | 100.0 |
| 7 | 100.8 | 100.4 | 96.5 |
| 14 | 100.6 | 99.4 | 92.0 |
| 29 | 99.8 | 98.8 | 82.3 |
| pH 9 | | | |
| 0 | 100.0 | 100.0 | 100.0 |
| 7 | 100.4 | 100.5 | 100.1 |
| 14 | 100.8 | 99.4 | 98.7 |
| 29 | 100.4 | 100.4 | 96.5 |

These results show that FMdC is very unstable at low pH (pH 1 and 3), degrading rapidly even when stored under refrigeration. It is considerably more stable at pH 5, although it clearly will not achieve the desired 18 months storage with no more than 10% degradation, even under refrigeration. At pH 7 the drug is considerably more stable at the one-month storage point; little degradation is seen at 5° or 25° C. and accelerated conditions (45° C.) are needed to bring about significant degradation over this time. The drug is clearly most stable at pH 9, where even under accelerated conditions (45° C.) degradation after 1 month is slight.

### Example 2

In this study the stabilty of FMdC glutamate and glycine buffers at pH 8.5 to 9.5 was evaluated at various storage conditions (5°, 25°, 35°, 45°, and 55° C.). FMdC solutions were made up at 1 mg/mL in 7.1 mg/mL monosodium glutamate monohydrate (38 mM) or 2.8 mg/mL glycine (37 mM) and pH adjusted to target with sodium hydroxide. Solution samples were placed in stoppered glass vials and stored at the various temperatures. FMdC content was assessed at various times using an HPLC method similar to that in Example 1: The column was a YMC Pack 5 μm ODS-A column, and the mobile phase was a 95/5 (v/v) sodium phosphate (pH 7)/acetonitrile. Detention was by UV at 268 nm. Results are summarized in Table 2 below:

TABLE 2
Stability of FMdC at pH 8.5 to 9.0 in Glutamate and Glycine Buffers

| | Glutamate | | Glutamate | | Glutamate | | Glycine | |
|---|---|---|---|---|---|---|---|---|
| Buffer | pH 8.5 | | pH 9.0 | | pH 9.5 | | pH 9.0 | |
| Time | FMdC | | FMdC | | FMdC | | FMdC | |
| (weeks) | (mg/mL) | pH | (mg/mL) | pH | (mglmL) | pH | (mg/ml) | pH |
| 5° C. | | | | | | | | |
| 0 | 1.00 | 8.50 | 1.01 | 9.00 | 1.00 | 9.50 | 1.00 | 9.00 |
| 12 | 1.00 | | 1.00 | | 1.00 | | 1.01 | |
| 16 | | 8.63 | | 9.10 | | 9.54 | | 9.30 |
| 24 | 1.00 | 8.66 | 1.00 | 9.05 | 1.01 | 9.57 | 1.00 | 9.33 |
| 25° C. | | | | | | | | |
| 0 | 1.00 | 8.50 | 1.01 | 9.00 | 1.00 | 9.50 | 1.00 | 9.00 |
| 4 | 1.00 | 8.55 | 1.01 | 9.07 | 1.00 | 9.53 | 1.01 | 9.33 |
| 16 | 0.99 | 7.93 | 0.99 | 8.80 | 1.00 | 9.55 | 1.00 | 9.30 |
| 24 | 0.99 | 8.64 | 0.99 | 9.09 | 0.99 | 9.57 | 0.99 | 9.32 |
| 35° C. | | | | | | | | |
| 0 | 1.00 | 8.50 | 1.01 | 9.00 | 1.00 | 9.50 | 1.00 | 9.00 |
| 4 | 1.00 | 8.55 | 1.01 | 9.06 | 1.00 | 9.54 | | 9.32 |
| 16 | 0.97 | 8.59 | 0.98 | 9.08 | 0.98 | 9.59 | 0.98 | 9.32 |
| 24 | 0.95 | 8.58 | 0.98 | 9.08 | 0.97 | 9.62 | 0.97 | 9.31 |
| 45° C. | | | | | | | | |
| 0 | 1.00 | 8.50 | 1.01 | 9.00 | 1.00 | 9.50 | 1.00 | 9.00 |
| 4 | 0.98 | 8.56 | 0.99 | 9.04 | 0.99 | 9.56 | 0.99 | 9.30 |
| 16 | 0.85 | 8.28 | 0.92 | 9.06 | 0.92 | 9.68 | 0.92 | 9.29 |
| 24 | 0.47 | 5.75 | 0.88 | 9.08 | 0.89 | 9.62 | 0.88 | 9.31 |
| 55° C. | | | | | | | | |
| 0 | 1.00 | 8.50 | 1.01 | 9.00 | 1.00 | 9.50 | 1.00 | 9.00 |
| 2 | 0.91 | | 0.96 | | 0.97 | | | |
| 4 | 0.72 | 7.56 | 0.91 | 9.01 | 0.93 | 9.62 | 0.91 | 9.27 |
| 8 | 0.03 | | 0.78 | | 0.82 | | 0.79 | |
| 16 | | | 0.60 | 8.44 | 0.69 | 9.97 | 0.63 | 8.92 |

These data show that FMdC is stable for almost 6 months at 5° C. or 25° C. in all four systems. After 24 weeks at 35° C., there is only a small amount of degradation, with very slightly more in the pH 8.5 glutamate buffer. At 45° C. differences between the systems become more apparent, with the pH 8.5 glutamate system exhibiting poorer stability than the other three. This trend is also demonstrated at 55° C., and also that the pH 9.5 glutamate system is somewhat more stable than the two pH 9.0 systems. Interestingly, the pH 9.0 glutamate system and the pH 9.0 glycine system exhibit very similar stability behavior.

The higher-temperature data can be evaluated further by Arrhenius analysis to predict shelf lives at lower temperatures. First-order rate constants are determined by fitting the concentration and time data to an equation of the form C(t)=C(0)·exp (−k·t), where C(t) is the concentration at time t, C(0) is the concentration at time 0, t is the time, and k is the first-order rate constant. The rate constants are in turn fitted to an equation of the form k (T)=A·exp (−Eₐ/RT), where k(T) is the rate constant at temperature T, Eₐ is the energy of activation, R is the gas constant, T is the absolute temperature (in ° K), and A is a constant. The Arrhenius plot (In k vs 1/T) for the data on the two pH 9.0 buffer systems yields a straight line, which extrapolates to provide a predicted rate constant at 25° C. of 3.0·10⁻⁴ week⁻¹, which corresponds to a shelf-life (time at which 5% degradation would be expected) at 25° C. of several years.

### Example 3

In this study the stability of FMdC at various concentrations in 100 mM sodium phosphate pH 9 was studied. FMdC solutions were made up and pH adjusted to target with sodium hydroxide. Solution samples were placed in stoppered glass vials and stored at the various temperatures. FMdC content was assessed at various times using an HPLC method similar to that in Example 2: the column was a YMC Pack 5 μm ODS-A column, and the mobile phase was 95/5 (v/v) sodium phosphate (pH 7)/acetonitrile. Flow rate was 1.0 mL/min. FMdC solution samples were diluted in mobile phase to a nominal FMdC concentration of 80 μg/mL, then injected onto the column in a volume of 10 μL. Detection was by UV at 268 nm. Results are summarized in Table 3 below:

TABLE 3
Two Year Stability for FMdC Formulations

| FMdC 2 mg/mL | | | FMdC 10 mg/mL | | |
|---|---|---|---|---|---|
| | FMdC | | Time (mon) | FMdC | |
| Time (mon) | (% of initial) | pH | (% of initial) | (% of initial) | pH |
| 50° C. | | | | | |
| 0 | 100.0 | 9.18 | 0 | 100.0 | 9.29 |
| 0.5 | 100.0 | 8.84 | 0.3 | 99.6 | 8.75 |
| 1.0 | 97.4 | 8.84 | 0.7 | 94.4 | 8.24 |
| 2.0 | 88.7 | 8.39 | 0.9 | 89.5 | 7.91 |
| 3.0 | 80.4 | 8.16 | 1.4 | 82.0 | 7.52 |
| | | | 2.1 | 67.4 | 7.01 |
| 40° C. | | | | | |
| 0 | 100.0 | 9.18 | 0 | 100.0 | 9.29 |
| 3 | 97.0 | 8.89 | 2.0 | 98.1 | 8.60 |
| 6 | 92.5 | 8.55 | 3.0 | 95.8 | 8.40 |
| 9 | 88.4 | 8.44 | 3.7 | 96.9 | 8.19 |
| | | | 4.1 | 94.9 | 8.08 |
| 25° C. | | | | | |
| 0 | 100.0 | 9.18 | 0 | 100.0 | 9.29 |
| 6 | 100.0 | 9.00 | 2.8 | 98.9 | 9.25 |
| 9 | 100.5 | 9.03 | 4.0 | 100.4 | 9.17 |
| 20 | 100.0 | 9.00 | 6.5 | 99.3 | 9.09 |
| | | | 7.9 | 98.1 | 9.06 |
| | | | 16 | 97.3 | 8.73 |
| | | | 23 | 97.1 | 8.74 |

These data clearly demonstrates that these systems are stable for over eighteen months at 25° C. and will likely be stable for well over two years at this temperature.

### Example 4

In this study the stability of FMdC solution 10 mg/mL was studied in various buffer systems. FMdC solutions were made up and pH adjusted to target with sodium hydroxide. Solution samples were placed in stoppered glass vials and stored at 40° C. FMdC content was assessed at various times using the HPLC method described in Example 3 above. Results are shown in Table 4 below:

TABLE 4
Various Buffer Systems for FMdC Formulations

| | | FMdC Content | | |
|---|---|---|---|---|
| Buffer system | Time (mon) | mg/mL | (% of initial) | pH |
| 100 mM | 0 | 10.1 | (100.0) | 9.44 |
| sodium | | | | |
| phosphate | 1 | 9.97 | (98.7) | 9.14 |
| | 2 | 9.66 | (95.6) | 8.69 |
| | 3 | 9.68 | (95.8) | 8.47 |
| | 4 | 9.24 | (91.5) | 8.18 |
| | 6 | 8.75 | (86.6) | 7.80 |
| | 9 | 6.40 | (63.4) | 7.14 |
| 20 mM sodium | 0 | 9.90 | (100.0) | 9.36 |
| phosphate | 1 | 10.1 | (101.6) | 8.32 |
| | 2 | 9.51 | (96.1) | 7.76 |
| | 3 | 9.10 | (91.9) | 7.72 |
| | 4.7 | 0.0 | (0.0) | 4.65 |
| 20 mM glycine | 0 | 10.2 | (100.0) | 9.60 |
| | 1 | 10.1 | (99.0) | 9.32 |
| | 2 | 9.66 | (94.7) | 9.24 |
| | 3 | 9.70 | (95.1) | 9.11 |
| | 4 | 9.49 | (93.0) | 8.86 |
| 20 mM TRIS | 0 | 10.1 | (100.0) | 9.42 |
| | 1 | 10.0 | (99.0) | 9.24 |
| | 2 | 9.82 | (97.2) | 9.05 |
| | 3 | 9.77 | (96.7) | 8.89 |
| | 4 | 9.46 | (93.7) | 8.58 |

These data shows glycine and TRIS are superior to phosphate, at the same buffer concentrations, in stabilizing FMdC. They also show that higher buffer concentrations (e.g. 100 mM phosphate) may be more desirable than lower concentrations (e.g., 20 mM phosphate).

We claim:

1. An aqueous composition comprising:
a) water;
b) tezacitabine; and
c) a buffer which maintains the pH of the composition at about 7 or above.

2. The composition of claim 1, wherein said buffer is selected from the group consisting of TRIS, glycine, glutamate and phosphate.

3. The composition of claim 1, wherein said buffer has a pH from 8 to 11.

4. The composition of claim 1, wherein said buffer has a pH from 8.5 to 10.0.

5. The composition of claim 1, wherein the concentration of tezacitabine in the composition is about 0.5 mg/mL to about 80 mg/mL.

6. The composition of claim 1, wherein the water is sterile water.

7. A method for enhancing the shelf life an aqueous solution of tezacitabine which said method comprises:
a) preparing an aqueous solution of tezacitabine; and
b) combining a sufficient amount of a buffer to the solution prepared in a) above to maintain the pH of the resulting solution at least 7 or above.

8. The method of claim 7, wherein said buffer is selected from the group consisting of TRIS, glycine, glutamate, and phosphate.

9. The method of claim 7, wherein the buffer is selected such as to provide a pH of the resulting solution from 8 to 11.

10. The method of claim 8, wherein the buffer is selected such as to provide a pH of the resulting solution from 8.5 to 10.

11. The method of claim 7, wherein the concentration of tezacitabine in the composition is about 2 mg/mL to 80 mg/mL.

12. The method of claim 7, wherein said method further comprises storing the stable liquid composition at a temperature below about 40° C.

13. The method of claim 12, wherein said temperature is about 25° C.
